(19) United States
(12) Patent Application Publication
(10) Pub. No.: US 2023/0277845 A1
(43) Pub. Date: Sep. 7, 2023

(54) ADJUSTABLE EXTENSION FOR MEDICAL IMPLANT

(71) Applicant: Cochlear Limited, Macquarie University, NSW (AU)

(72) Inventors: Wim Bervoets, Mechelen (BE); Folkert Seeba, Hannover (DE); Koen Erik Van den Heuvel, Mechelen (BE)

(21) Appl. No.: 18/000,202

(22) PCT Filed: May 10, 2021
(86) PCT No.: PCT/IB2021/053960

§ 371 Date: Nov. 29, 2022

Related U.S. Application Data
(60) Provisional application No. 63/042,201, filed on Jun. 22, 2020.

(51) Int. Cl. A61N 1/36; A61F 2/18

(52) U.S. Cl. CPC A61N 1/36038; A61F 2/18

(57) ABSTRACT

An apparatus includes a transducer, a first element, and a second element configured to be at least partially implanted on or within a recipient. The transducer is in mechanical communication with a first portion of the recipient’s body, the first element is configured to be in mechanical communication with the transducer and includes an orifice having a first length and extending from a first surface of the first element to a second surface of the first element. The second element includes a first end and a second end with a second length therebetween, the second length longer than the first length. The second element is configured to extend through the orifice and, during implantation, to be slidably adjusted relative to the orifice and affixed to the first element such that the second end is in mechanical communication with a second portion of the recipient’s body.

### BACKGROUND

### Field

The present application relates generally to implantable medical prostheses, and more specifically to middle ear transducers (e.g., actuators; microphones) for implantable auditory prostheses.

### Description of the Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect disclosed herein, an apparatus comprises a transducer configured to be at least partially implanted on or within a recipient and in mechanical communication with a first portion of the recipient’s body. The apparatus further comprises a first element configured to be at least partially implanted on or within the recipient, the first element configured to be in mechanical communication with the transducer. The first element comprises an orifice having a first length and extending from a first surface of the first element to a second surface of the first element. The apparatus further comprises a second element configured to be at least partially implanted on or within the recipient. The second element comprises a first end and a second end with a second length therebetween, the second length longer than the first length. The second element is configured to extend through the orifice and, during implantation, to be slidably adjusted relative to the orifice and affixed to the first element such that the second end is in mechanical communication with a second portion of the recipient’s body.

In another aspect disclosed herein, an apparatus comprises a first portion configured to be attached to a portion of a recipient’s auditory system. The apparatus further comprises a second portion configured to be in mechanical communication with a transducer and with the first portion such that the second portion is configured to be rotatably adjusted in at least one direction relative to the first portion.

In another aspect disclosed herein, a method comprises at least partially implanting an assembly on or within a recipient such that the assembly is in mechanical communication with a first portion of a recipient’s body. The assembly comprises a transducer and a first element in mechanical communication with the transducer. The first element comprises a through-hole. The method further comprises adjusting a position of a second element within the through-hole such that second element extends out of both sides of the through-hole and the second element is in mechanical communication with a second portion of the recipient’s body. The method further comprises affixing the first element and the second element to one another such that mechanical vibrations generated by the transducer propagate through the first element and the second element to the second portion of the recipient’s body.

### BRIEF DESCRIPTION OF THE DRAWINGS

Implementations are described herein in conjunction with the accompanying drawings, in which:

FIG. 1 is a perspective view of an example cochlear implant auditory prosthesis implanted in a recipient in accordance with certain implementations described herein;

FIG. 2 is a perspective view of an example fully implantable middle ear implant auditory prosthesis implanted in a recipient in accordance with certain implementations described herein;

FIGS. 3A-3C schematically illustrate example prior middle ear transducer assemblies 300 that are based on a two-point fixation concept;

FIGS. 4A-4B and 5A-5B schematically illustrate two example apparatus in accordance with certain implementations described herein;

FIGS. 6A-6E schematically illustrate example first elements in accordance with certain implementations described herein;

FIGS. 7A-7F schematically illustrate example second elements in accordance with certain implementations described herein

FIGS. 8A-8B schematically illustrate an example apparatus in accordance with certain implementations described herein; and

FIG. 9 is a flow diagram of an example method in accordance with certain embodiments described herein.

### DETAILED DESCRIPTION

Sensorineural hearing loss (SNHL) is a permanent hearing loss due to damage that prevents or weakens nerve signals transmitted to the brain. Severe to profound SNHL can be addressed by a cochlear implant auditory prosthesis, while less severe SNHL can be addressed by an auditory prosthesis comprising a middle ear implant in contact with one of the ossicles of the ear, since patients with less severe SNHL can have a middle ear ossicular chain that is intact (e.g., capable of moving freely; not being blocked or having excessive conductive loss). Such middle ear implants follow the general practice of not removing functioning parts of the body unless necessary, and can be more effective for addressing less severe SNHL than are hearing aids or auditory prostheses using bone-anchored implants (e.g., which can utilize large amplification levels which can result in feedback issues).

Conductive hearing loss (CHL) is due to obstruction or damage to the outer ear or middle ear that prevents sound from being conducted to the inner ear. CHL can be addressed by an auditory prosthesis comprising a bone-anchored hearing aid that transmits sound vibrations to travel through the skull bone to the inner ear, thereby bypassing the middle ear ossicles and tympanic membrane. Such bone-anchored hearing aids can be more effective for many CHL patients than are hearing aids that are positioned within the ear canal (e.g., which can utilize large amplification levels which can result in feedback issues).

Mixed hearing loss (MHL) is any combination of SNHL and CHL, and can be addressed by an auditory prosthesis comprising a bone-anchored hearing aid or comprising a middle ear implant (e.g., to address the SNHL component). However, in contrast to addressing solely SNHL, addressing MHL can comprise removal and replacement or bypass (e.g., by extension or prosthesis) of part of the ossicular chain to address the CHL component.

Certain implementations described herein provide a transducer assembly comprising an extension (e.g., rod; wire) configured to be adjustably slid through an orifice (e.g., of a hollow drive pin) to controllably adjust a length of the transducer assembly between two fixation points while the medical practitioner (e.g., surgeon) is implanting the transducer assembly and establishing a mechanical coupling between the transducer assembly and a portion of the recipient’s body. Certain implementations described herein are configured to affix the extension within the orifice (e.g., by crimping the hollow drive pin) at a location behind the transducer (e.g., at a more easily accessible position that is also sufficiently spaced away from sensitive structures of the recipient’s body to reduce the probability of mishap during the affixation process).

The teachings detailed herein are applicable, in at least some implementations, to any type of auditory prosthesis utilizing an implantable transducer assembly including but not limited to: electro-acoustic electrical/acoustic systems, cochlear implant devices, implantable hearing aid devices, middle ear implant devices, bone conduction devices (e.g., active bone conduction devices; passive bone conduction devices, percutaneous bone conduction devices; transcutaneous bone conduction devices), Direct Acoustic Cochlear Implant (DACI), middle ear transducer (MET), electro-acoustic implant devices, other types of auditory prosthesis devices, and/or combinations or variations thereof, or any other suitable hearing prosthesis system with or without one or more external components. Implementations can include any type of auditory prosthesis that can utilize the teachings detailed herein and/or variations thereof. Certain such implementations can be referred to as “partially implantable,” “semi-implantable,” “mostly implantable,” “fully implantable,” or “totally implantable” auditory prostheses. In some implementations, the teachings detailed herein and/or variations thereof can be utilized in other types of prostheses beyond auditory prostheses.

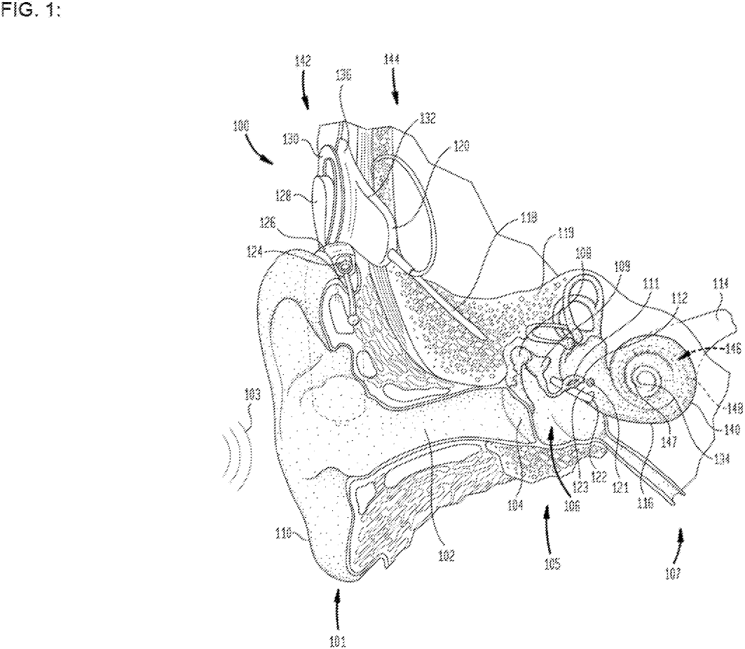

FIG. 1 is a perspective view of an example cochlear implant auditory prosthesis 100 implanted in a recipient in accordance with certain implementations described herein. The example auditory prosthesis 100 is shown in FIG. 1 as comprising an implanted stimulator unit 120 (e.g., an actuator) and a microphone assembly 124 that is external to the recipient (e.g., a partially implantable cochlear implant). An example auditory prosthesis 100 (e.g., a totally implantable cochlear implant; a mostly implantable cochlear implant) in accordance with certain implementations described herein can replace the external microphone assembly 124 shown in FIG. 1 with a subcutaneously implantable microphone assembly 124, as described more fully herein.

As shown in FIG. 1, the recipient has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

As shown in FIG. 1, the example auditory prosthesis 100 comprises one or more components which are temporarily or permanently implanted in the recipient. The example auditory prosthesis 100 is shown in FIG. 1 with an external component 142 which is directly or indirectly attached to the recipient’s body, and an internal component 144 which is temporarily or permanently implanted in the recipient (e.g., positioned in a recess of the temporal bone adjacent auricle 110 of the recipient). The external component 142 typically comprises one or more sound input elements (e.g., an external microphone 124) for detecting sound, a sound processing unit 126 (e.g., disposed in a Behind-The-Ear unit), a power source (not shown), and an external transmitter unit 128. In the illustrative implementations of FIG. 1, the external transmitter unit 128 comprises an external coil 130 (e.g., a wire antenna coil comprising multiple turns of electrically insulated single-strand or multi-strand platinum or gold wire) and, preferably, a magnet (not shown) secured directly or indirectly to the external coil 130. The external coil 130 of the external transmitter unit 128 is part of an inductive radio frequency (RF) communication link with the internal component 144. The sound processing unit 126 processes the output of the microphone 124 that is positioned externally to the recipient’s body, in the depicted implementation, by the recipient’s auricle 110. The sound processing unit 126 processes the output of the microphone 124 and generates encoded signals, sometimes referred to herein as encoded data signals, which are provided to the external transmitter unit 128 (e.g., via a cable). As will be appreciated, the sound processing unit 126 can utilize digital processing techniques to provide frequency shaping, amplification, compression, and other signal conditioning, including conditioning based on recipient-specific fitting parameters.

The power source of the external component 142 is configured to provide power to the auditory prosthesis 100, where the auditory prosthesis 100 includes a battery (e.g., located in the internal component 144, or disposed in a separate implanted location) that is recharged by the power provided from the external component 142 (e.g., via a transcutaneous energy transfer link). The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power stored by the rechargeable battery is distributed to the various other implanted components as needed.

The internal component 144 comprises an internal receiver unit 132, a stimulator unit 120, and an elongate electrode assembly 118. In some implementations, the internal receiver unit 132 and the stimulator unit 120 are hermetically sealed within a biocompatible housing. The internal receiver unit 132 comprises an internal coil 136 (e.g., a wire antenna coil comprising multiple turns of electrically insulated single-strand or multi-strand platinum or gold wire), and preferably, a magnet (also not shown) fixed relative to the internal coil 136. The internal receiver unit 132 and the stimulator unit 120 are hermetically sealed within a biocompatible housing, sometimes collectively referred to as a stimulator/receiver unit. The internal coil 136 receives power and/or data signals from the external coil 130 via a transcutaneous energy transfer link (e.g., an inductive RF link). The stimulator unit 120 generates electrical stimulation signals based on the data signals, and the stimulation signals are delivered to the recipient via the elongate electrode assembly 118.

The elongate electrode assembly 118 has a proximal end connected to the stimulator unit 120, and a distal end implanted in the cochlea 140. The electrode assembly 118 extends from the stimulator unit 120 to the cochlea 140 through the mastoid bone 119. In some implementations, the electrode assembly 118 may be implanted at least in the basal region 116, and sometimes further. For example, the electrode assembly 118 may extend towards apical end of cochlea 140, referred to as cochlea apex 134. In certain circumstances, the electrode assembly 118 may be inserted into the cochlea 140 via a cochleostomy 122. In other circumstances, a cochleostomy may be formed through the round window 121, the oval window 112, the promontory 123, or through an apical turn 147 of the cochlea 140.

The elongate electrode assembly 118 comprises a longitudinally aligned and distally extending array 146 of electrodes or contacts 148, sometimes referred to as electrode or contact array 146 herein, disposed along a length thereof. Although the electrode array 146 can be disposed on the electrode assembly 118, in most practical applications, the electrode array 146 is integrated into the electrode assembly 118 (e.g., the electrode array 146 is disposed in the electrode assembly 118). As noted, the stimulator unit 120 generates stimulation signals which are applied by the electrodes 148 to the cochlea 140, thereby stimulating the auditory nerve 114.

While FIG. 1 schematically illustrates an auditory prosthesis 100 utilizing an external component 142 comprising an external microphone 124, an external sound processing unit 126, and an external power source, in certain other implementations, one or more of the microphone 124, sound processing unit 126, and power source are implantable on or within the recipient (e.g., within the internal component 144). For example, the auditory prosthesis 100 can have each of the microphone 124, sound processing unit 126, and power source implantable on or within the recipient (e.g., encapsulated within a biocompatible assembly located subcutaneously), and can be referred to as a totally implantable cochlear implant (“TICI”). For another example, the auditory prosthesis 100 can have most components of the cochlear implant (e.g., excluding the microphone, which can be an in-the-ear-canal microphone) implantable on or within the recipient, and can be referred to as a mostly implantable cochlear implant (“MICI”).

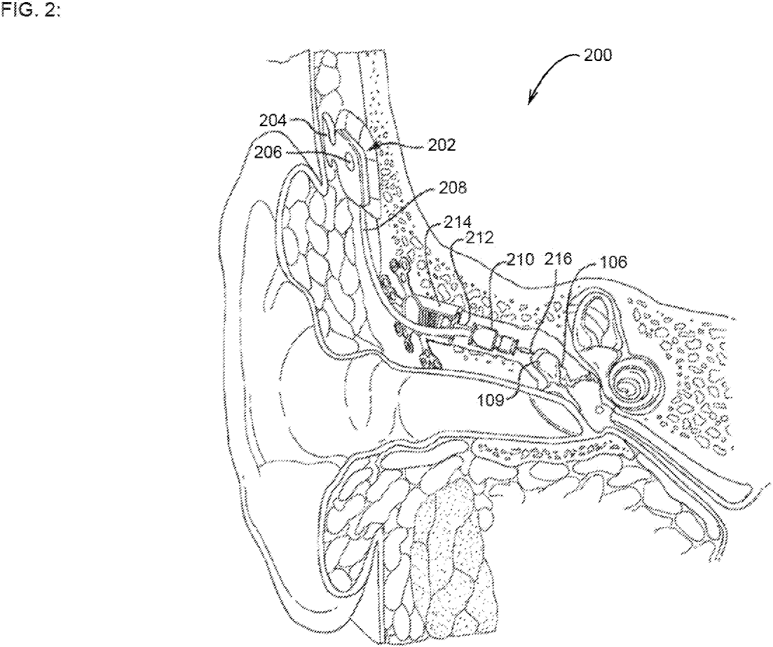

FIG. 2 schematically illustrates a perspective view of an example fully implantable auditory prosthesis 200 (e.g., fully implantable middle ear implant or totally implantable acoustic system), implanted in a recipient, utilizing an acoustic actuator in accordance with certain implementations described herein. The example auditory prosthesis 200 of FIG. 2 comprises a biocompatible implantable assembly 202 (e.g., comprising an implantable capsule) located subcutaneously (e.g., beneath the recipient’s skin and on a recipient’s skull). While FIG. 2 schematically illustrates an example implantable assembly 202 comprising a microphone, in other example auditory prostheses 200, a pendant microphone can be used (e.g., connected to the implantable assembly 202 by a cable). The implantable assembly 202 includes a signal receiver 204 (e.g., comprising a coil element) and an acoustic transducer 206 (e.g., a microphone comprising a diaphragm and an electret or piezoelectric transducer) that is positioned to receive acoustic signals through the recipient’s overlying tissue. The implantable assembly 202 may further be utilized to house a number of components of the fully implantable auditory prosthesis 200. For example, the implantable assembly 202 can include an energy storage device and a signal processor (e.g., a sound processing unit). Various additional processing logic and/or circuitry components can also be included in the implantable assembly 202 as a matter of design choice.

For the example auditory prosthesis 200 shown in FIG. 2, the signal processor of the implantable assembly 202 is in operative communication (e.g., electrically interconnected via a wire 208) with an actuator 210 (e.g., comprising a transducer configured to generate mechanical vibrations in response to electrical signals from the signal processor). In certain implementations, the example auditory prosthesis 100, 200 shown in FIGS. 1 and 2 can comprise an implantable microphone assembly, such as the microphone assembly 206 shown in FIG. 2. For such an example auditory prosthesis 100, the signal processor of the implantable assembly 202 can be in operative communication (e.g., electrically interconnected via a wire) with the microphone assembly 206 and the stimulator unit of the main implantable component 120. In certain implementations, at least one of the microphone assembly 206 and the signal processor (e.g., a sound processing unit) is implanted on or within the recipient.

The actuator 210 of the example auditory prosthesis 200 shown in FIG. 2 is supportably connected to a positioning system 212, which in turn, is connected to a bone anchor 214 mounted within the recipient’s mastoid process (e.g., via a hole drilled through the skull). The actuator 210 includes a connection apparatus 216 for connecting the actuator 210 to the ossicles 106 of the recipient. In a connected state, the connection apparatus 216 provides a communication path for acoustic stimulation of the ossicles 106 (e.g., through transmission of vibrations from the actuator 210 to the incus 109).

During normal operation, ambient acoustic signals (e.g., ambient sound) impinge on the recipient’s tissue and are received transcutaneously at the microphone assembly 206. Upon receipt of the transcutaneous signals, a signal processor within the implantable assembly 202 processes the signals to provide a processed audio drive signal via wire 208 to the actuator 210. As will be appreciated, the signal processor may utilize digital processing techniques to provide frequency shaping, amplification, compression, and other signal conditioning, including conditioning based on recipient-specific fitting parameters. The audio drive signal causes the actuator 210 to transmit vibrations at acoustic frequencies to the connection apparatus 216 to affect the desired sound sensation via mechanical stimulation of the incus 109 of the recipient.

The subcutaneously implantable microphone assembly 202 is configured to respond to auditory signals (e.g., sound; pressure variations in an audible frequency range) by generating output signals (e.g., electrical signals; optical signals; electromagnetic signals) indicative of the auditory signals received by the microphone assembly 202, and these output signals are used by the auditory prosthesis 100, 200 to generate stimulation signals which are provided to the recipient’s auditory system. To compensate for the decreased acoustic signal strength reaching the microphone assembly 202 by virtue of being implanted, the diaphragm of an implantable microphone assembly 202 is configured to provide higher sensitivity than are external non-implantable microphone assemblies (e.g., by using diaphragms that are larger than diaphragms for external non-implantable microphone assemblies).

The example auditory prostheses 100 shown in FIG. 1 utilizes an external microphone 124 and the auditory prosthesis 200 shown in FIG. 2 utilizes an implantable microphone assembly 206 comprising a subcutaneously implantable acoustic transducer. In certain implementations described herein, the auditory prosthesis 100 utilizes one or more implanted microphone assemblies on or within the recipient. In certain implementations described herein, the auditory prosthesis 200 utilizes one or more microphone assemblies that are positioned external to the recipient and/or that are implanted on or within the recipient, and utilizes one or more acoustic transducers (e.g., actuator 210) that are implanted on or within the recipient. In certain implementations, an external microphone assembly can be used to supplement an implantable microphone assembly of the auditory prosthesis 100, 200. Thus, the teachings detailed herein and/or variations thereof can be utilized with any type of external or implantable microphone arrangement, and the acoustic transducers shown in FIGS. 1 and 2 are merely illustrative.

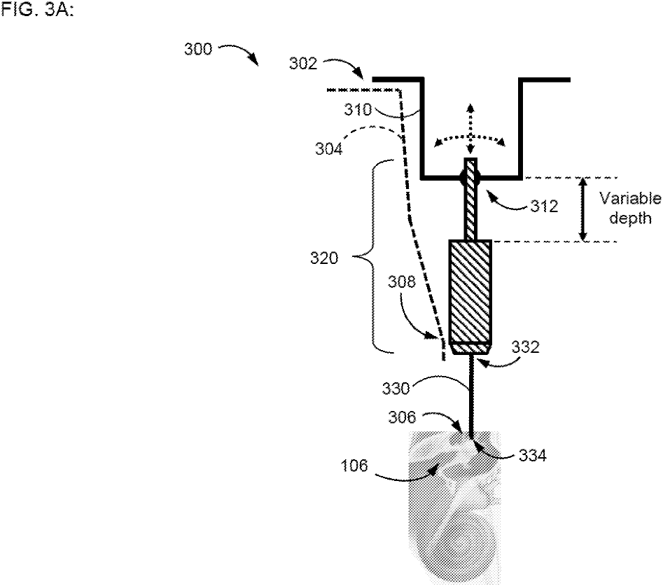

FIGS. 3A-3C schematically illustrate example prior middle ear transducer assemblies 300 that are based on a two-point fixation concept with one fixation point 302 at a surface of the recipient’s skull 304 and a second fixation point 306 at a middle ear target (e.g., an ossicle 106; incus 109). The transducer assembly 300 bridges the physical gap between the two fixation points 302, 306 and comprises a fixation element 310 (e.g., bracket) that is affixed to the recipient’s skull 304 and a transducer 320 in mechanical communication with the fixation element 310. The transducer 320 comprises a connection apparatus 330 (e.g., connection apparatus 216) having a first end 332 in mechanical communication with the transducer 320 and a second end 334 in mechanical communication with the middle ear target. The connection apparatus 330 is configured to conduct mechanical vibrations from the transducer 320 to the middle ear target.

The transducer assembly 300 further includes a linear motion mechanism (not shown) (e.g., z-adjustment microdrive and compression unit) that is configured to mechanically couple the transducer 320 to the fixation element 310 and to controllably adjust a linear position (e.g., depth) of the transducer 320 (denoted in FIGS. 3A-3C by a vertical dotted line) allowing for a z- adjustment microdrive movement of about 4 to 5 millimeters, in addition to a gross translation of the transducer 320 sliding through the compression unit (e.g., by a distance of about 4 to 5 millimeters). In addition, the transducer assembly 300 further comprises a rotatable coupler 312 configured to adjust an angle of the transducer 320 relative to the fixation element 310 (denoted in FIGS. 3A-3C by a curved dotted line).

The first end 332 of the connection apparatus 330 (e.g., connection apparatus 216) can comprise a solid first rod extending from a front portion of the transducer 320 and a hollow tube welded onto the first rod, forming a blind hole (e.g., 2 millimeters deep) configured to receive a first end of a solid second rod, the second rod comprising a second end that is the second end 334 of the connection apparatus 330. During an implantation process for the transducer assembly 300, the transducer 320 is positioned (e.g., at a depth selected to avoid the transducer 320 from contacting or being interfered by other bone portions 308 of the skull 304). A length measurement is made (e.g., using a template device) to determine a distance between the transducer 320 and the middle ear target (e.g., a distance between an inner surface of the blind hole of the transducer 320 into which the first end of the second rod is to be inserted), and the second rod is cut to an appropriate length using a cutting tool (e.g., on the operating room table), after which the first end of the second rod is positioned and affixed to the transducer 320 (e.g., the blind hole crimped onto the first end of the second rod), the transducer 320 is inserted and fixed to the fixation element 310, and the second end of the second rod is attached to the middle ear target. This process risks errors with the handling, cutting, and positioning of the second rod. Using connection apparatus 330 with pre-fixed lengths (e.g., a “one size fits all”) may not be recommended as adequate statistical data of the middle ear variability would be required and would result in design compromises. In addition, a pre-fixed connection apparatus 330 would be logistically complex to accommodate for the middle ear coupling variant options. FIGS. 3B and 3C schematically illustrate the second end 332 of the connection apparatus 330 having a structure configured to be affixed to the ossicles 106 in a manner compatible with use in addressing SNHL and MHL, respectively.

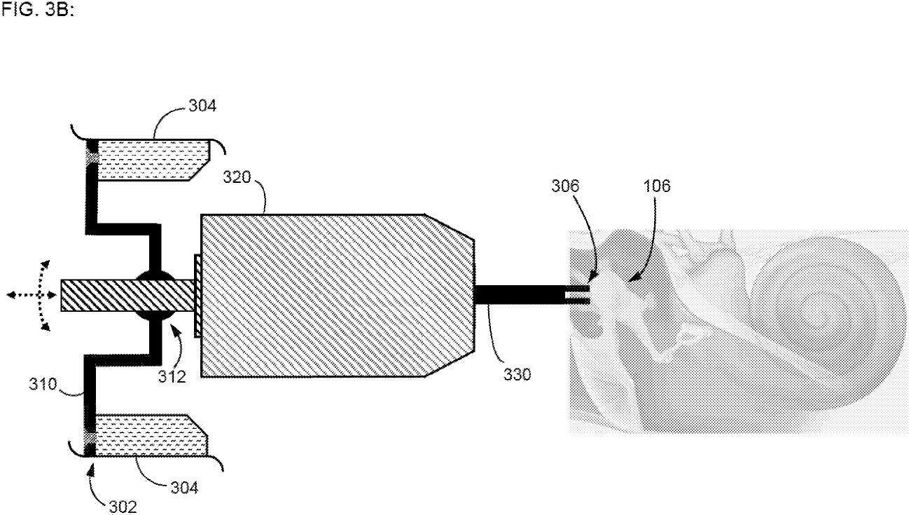

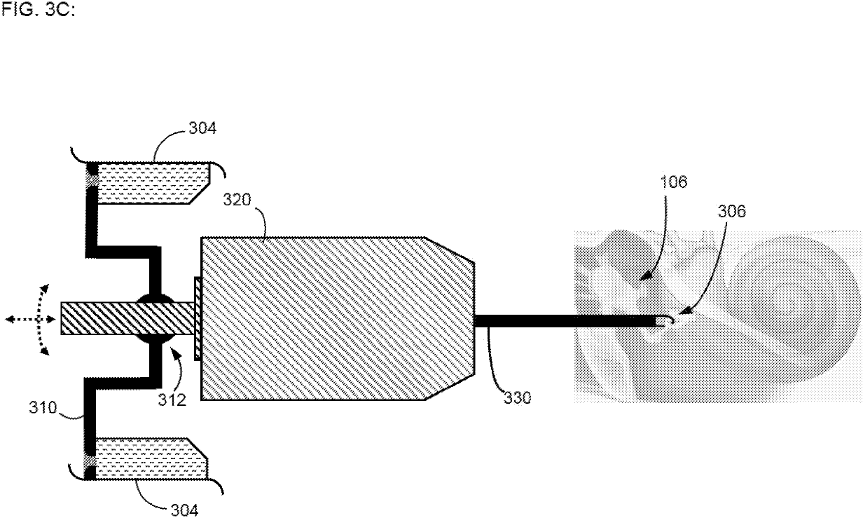

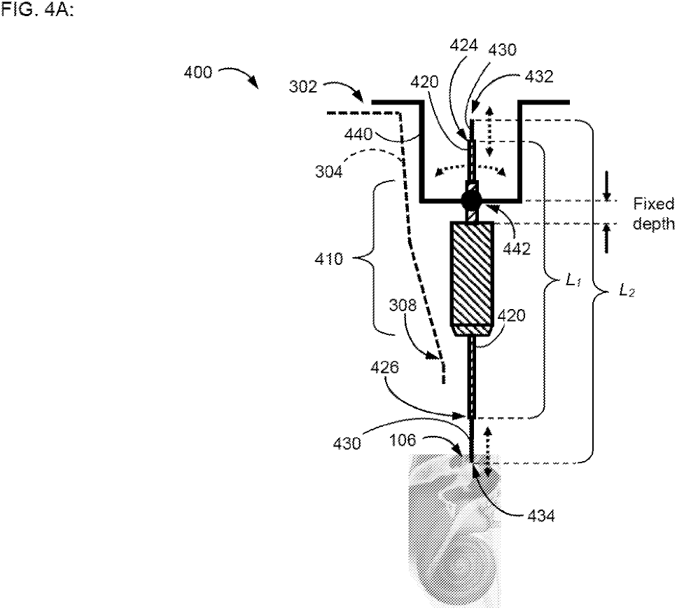

FIGS. 4A-4B and 5A-5B schematically illustrate two example apparatus 400 in accordance with certain implementations described herein. The apparatus 400 comprises a transducer 410 configured to be at least partially implanted on or within a recipient and in mechanical communication with a first portion of the recipient’s body (e.g., a skull 304). The apparatus 400 further comprises a first element 420 configured to be at least partially implanted on or within the recipient. The first element 420 is configured to be in mechanical communication with the transducer 410. The first element 420 comprises an orifice 422 having a first length L₁ and extending from a first surface 424 of the first element 420 to a second surface 426 of the first element 420 (see, e.g., FIGS. 4B and 5B). The apparatus 400 further comprises a second element 430 configured to be at least partially implanted on or within the recipient. The second element 430 comprises a first end 432 and a second end 434 with a second length L₂ therebetween. The second length L₂ is longer than the first length L₁. The second element 430 is configured to extend through the orifice 422 and, during implantation, to be slidably adjusted (e.g., in a substantially continuous motion or in discrete steps) relative to the orifice 422 and affixed to the first element 420 such that the second end 434 is in mechanical communication with a second portion of the recipient’s body (e.g., an ossicle 106; incus 109). For example, at least a portion of the first element 420 and at least a portion of the second element 430 can be configured to be implanted within a middle ear region of the recipient’s body, and the second end 434 of the second element 430 can be configured to be in mechanical communication with an ossicle 106, a portion of the cochlea 140, the otic capsule, or the semicircular canal of the recipient’s body.

In certain implementations, as schematically illustrated by FIGS. 4A-4B and 5A-5B, the transducer 410 comprises an actuator (e.g., an actuator 210 of a middle ear auditory prosthesis 200) configured to generate mechanical vibrations in response to electrical signals from a signal processor. For example, as schematically illustrated by FIGS. 4A-4B and 5A-5B, the actuator is mechanically connected to a fixation element 440 (e.g., bracket) that is mechanically connected (e.g., anchored) to the recipient’s skull 304 (e.g., via a hole drilled through the skull 304). In certain other implementations, the transducer 410 comprises an implantable microphone assembly (e.g., a tubular microphone assembly of an auditory prosthesis) configured to be in mechanically communication with an ossicle 106 (e.g., incus 109) and responsive to mechanical vibrations of the ossicle 106 corresponding to auditory signals received by the recipient’s tympanic membrane by generating output signals (e.g., electrical signals; optical signals; electromagnetic signals) indicative of the auditory signals.

In certain implementations, the fixation element 440 includes a coupler 442 configured to adjustably connect the transducer 410 to the fixation element 440. For example, the coupler 442 can comprise an adjustably rotatable coupler (e.g., ball and socket, as schematically illustrated by FIG. 4B) or an adjustably bendable coupler (e.g., bendable portion of the fixation element 440 with the transducer 410 welded to the fixation element 440). The adjustably rotatable coupler is configured to controllably adjust an angle of the transducer 410 relative to the fixation element 440 (denoted in FIG. 4A by a curved dotted line). The fixation element 440 of certain implementations is configured to hold the transducer 410 at a fixed depth relative to the fixation element 440. Thus, in contrast to the transducer assemblies 300 of FIGS. 3A-3C, the fixation element 440 of certain implementations does not include a linear motion mechanism (e.g., microdrive and compression unit) configured to controllably adjust a linear position (e.g., depth) of the transducer 410. In certain implementations, the fixation element 440 can comprise a drive that provides fine adjustment motion (e.g., less than or equal to 1 millimeter). For example, the fine adjustment motion can be used to generate a desired pre-load between the second element 430 and the second portion of the recipient’s body (e.g., the ossicles 106).

In certain implementations, as schematically illustrated by FIGS. 4A-4B, the first element 420 comprises a hollow cylindrical tube (e.g., a hollow actuator drive pin comprising titanium) having an inner diameter (e.g., in a range of 0.1 millimeter to 1 millimeter) and having a length L₁ between the first surface 424 and the second surface 426 (e.g., in a range of 10 millimeters to 30 millimeters; in a range of 20 millimeters to 40 millimeters). The first element 420 can extend through the transducer 410 (e.g., along a longitudinal axis of the transducer 410; the first surface 424 and the second surface 426 outside the transducer 410) and through the coupler 442 of the fixation element 440. For a transducer 410 comprising an actuator (e.g., actuator 210), the first element 420 is configured to receive vibrations from the actuator, or for a transducer 410 comprising a microphone, the first element 420 is configured to transmit vibrations to the microphone. For example, the first element 420 can comprise at least one structure 427 (e.g., fin) extending outward from a longitudinal axis of the first element 420, as schematically illustrated by FIG. 4B, and configured to be mechanically affixed to the actuator/microphone of the transducer 410.

In certain implementations, as schematically illustrated by FIGS. 5A-5B, the first element 420 comprises an extension element (e.g., comprising titanium) having a first portion 510 extending along (e.g., substantially parallel to) a longitudinal axis of the transducer 410 and a second portion 520 extending substantially perpendicular to the first portion 510 and having a thickness L₁ between the first surface 424 and the second surface 426 (e.g., in a range of 0.15 millimeter to 1.5 millimeters). The first element 420 further comprises an orifice 422 extending through the second portion 520 from the first surface 424 to the second surface 426 (e.g., a through hole) with an inner diameter (e.g., in a range of 0.1 millimeter to 1 millimeter; in a range of 0.1 millimeter to 0.2 millimeter).

In certain implementations, as schematically illustrated by FIGS. 4A-4B and 5A-5B, the second element 430 comprises an elongate body having an outer diameter (e.g., in a range of 0.1 millimeter to 1 millimeter; in a range of 0.1 millimeter to 0.2 millimeter) and having a second length L₂ between the first end 432 of the second element 430 and the second end 434 of the second element 430 (e.g., in a range of 5 millimeters to 30 millimeters; in a range of 8 millimeters to 16 millimeters). In certain implementations, the second element 430 comprises a rigid rod (e.g., titanium), while in certain other implementations, the second element comprises a flexible wire. The second element 430 is configured to extend through the orifice 422 of the first element 420 (e.g., with the first end 432 and the second end 434 both outside the orifice 422) and to be slidably positioned within the orifice 422 (e.g., manipulated by the medical practitioner during implantation). For example, the second element 430 is configured to be slid within the orifice 422 along a distance within a range of 0.1 millimeter to 25 millimeters (e.g., 0.1 millimeter to 1 millimeter).

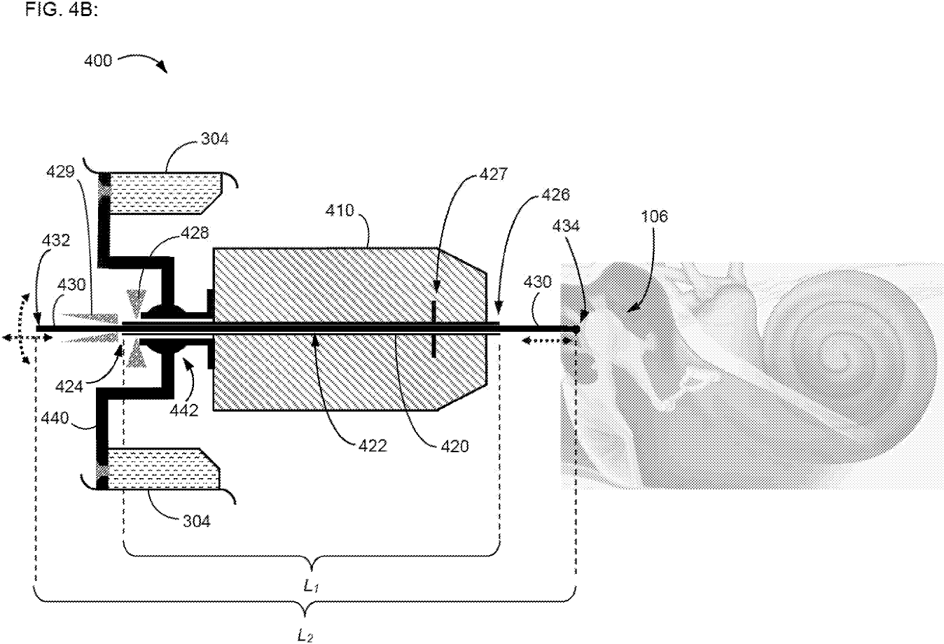

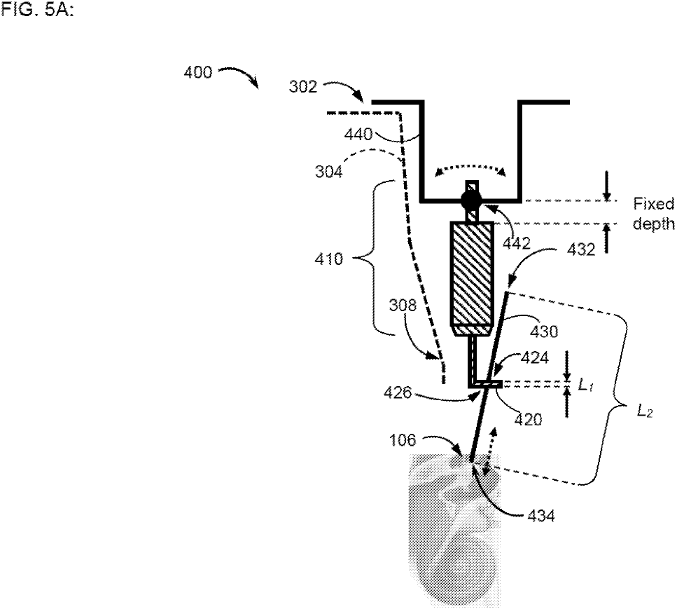

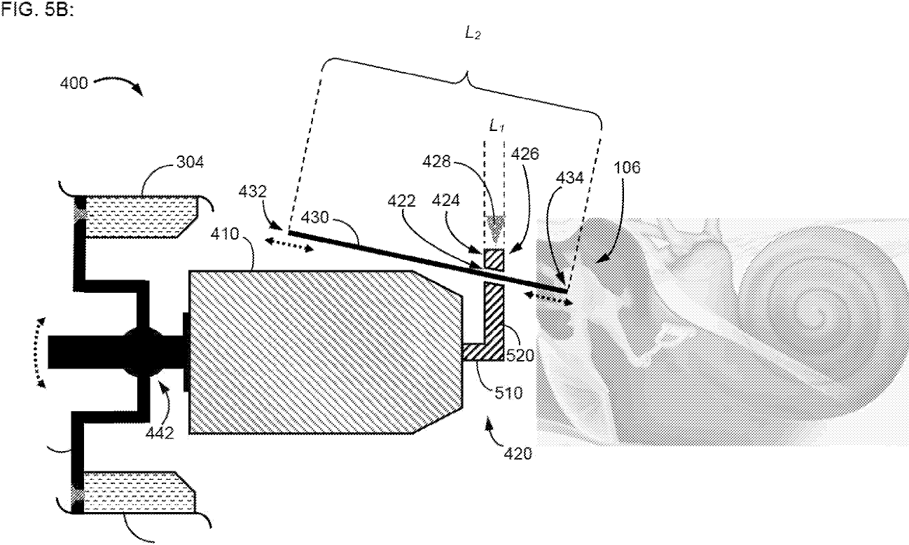

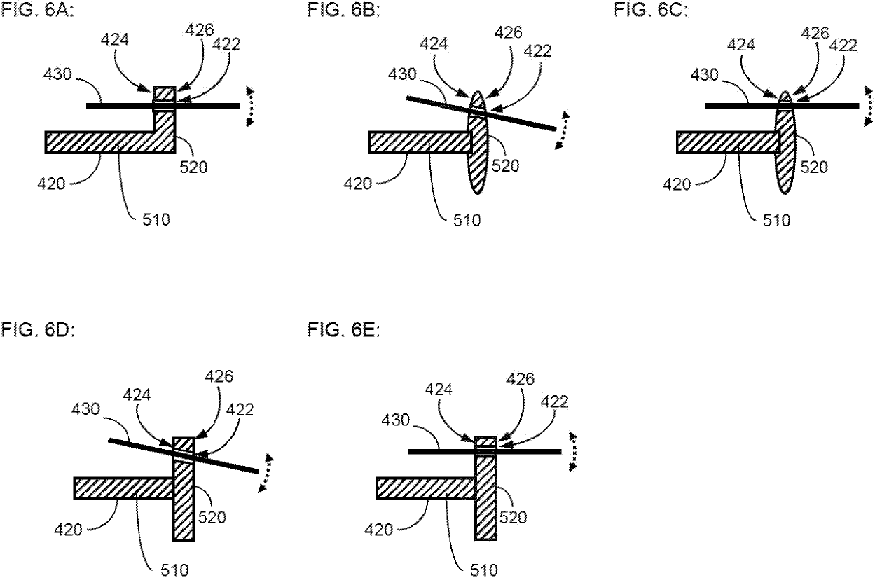

FIGS. 6A-6E schematically illustrate other example first elements 420 in accordance with certain implementations described herein. For example, for the “L-shaped” first elements 420 of FIGS. 5A-5B and 6A, the first surface 424 and the second surface 426 are substantially parallel to one another. The orifice 422 and the second element 430 of FIGS. 5A-5B are at a non-zero angle relative to the first portion 510 of the first element 420, while the orifice 422 and the second element 430 of FIG. 6A are substantially parallel to the longitudinal axis of the transducer 410. FIGS. 6B-6C schematically illustrate example first elements 420 with a “disk-shaped” second portion 520, and with the orifice 422 and the second element 430 substantially non-parallel and parallel, respectively, to the first portion 510 of the first element 420. FIGS. 6D-6E schematically illustrate example “T-shaped” first elements 420 with the first surface 424 and the second surface 426 parallel to one another, and with the orifice 422 and second element 430 substantially non-parallel and parallel, respectively, to the first portion 510 of the first element 420. Various other configurations of the first element 420 and the second element 430 are also compatible with certain implementations described herein. For example, the first element 420 can comprise multiple orifices 422 at various positions on the second portion 520 (e.g., on opposite sides of the first portion 510 shown in FIGS. 6B-6E). In certain implementations, the second element 430 is configured to be pivoted within the orifice 422 (e.g., shown by the dotted double arrows of FIGS. 6A-6E) prior to being fixed (e.g., crimped) within the orifice 422 (e.g., to provide adjustment of the angle between the first element 420 and the second element 430). In certain implementations, the orifice 422 comprises a slit extending to an edge of the second portion 520 (e.g., to a circumference of the disk-shaped second portion 520 of FIGS. 6B-6C). In certain implementations, the slit can comprise an integrated clip configured to affix the first element 420 and the second element 430 to one another.

In certain implementations, the first element 420 is configured to be affixed to the second element 430 once the second element 430 is positioned by the medical practitioner, as described more fully herein. As schematically illustrated by FIG. 4B, the first element 420 of certain implementations is configured to be crimped to the second element 430 by a feature 428 configured to crimp the first element 420 and/or the second element 430. In certain implementations, the first element 420 comprises the feature 428, while in certain other implementations, the feature 428 is a separate tool from the first element 420. For example, as schematically illustrated by FIG. 4B, the first element 420 can be crimped at a position proximate to the first surface 424 (e.g., with the transducer 410 between the crimped position and the second end 434 of the third element 430). The feature 428 of certain such implementations can comprise a cutting element (e.g., blade) configured to cut and remove an excess portion of the second element 430 extending past the crimped position, and this cutting element can be configured to cut the second element 430 with a cutting action that also performs the crimping action. Certain implementations further comprise a clip (e.g., a component of the first element 420 or of the second element 430, or a component separate from the first element 420 and the second element 430) that is configured to affix (e.g., clip) the first element 420 and the second element 430 to one another. Such a clip can be an alternative to crimping or using an adhesive and can be configured to be reversibly disengaged, if warranted, to make revision surgery easier.

In certain other implementations, the first element 420 is configured to be crimped at a position proximate to the second surface 426 (e.g., with the crimped position between the transducer 410 and the second end 434 of the third element 430). For example, as schematically illustrated by FIG. 5B, the feature 428 can be configured to crimp the second portion 520 of the first element 420 to affix the second element 430 within the orifice 422. In certain other implementations, other affixation techniques (e.g., applying adhesive to one or both of the first element 420 and the second element 430) besides crimping can be used.

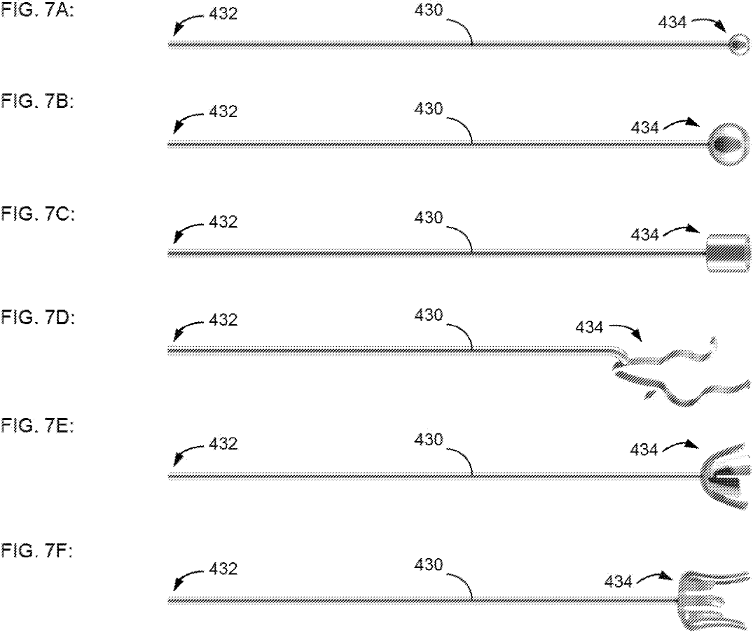

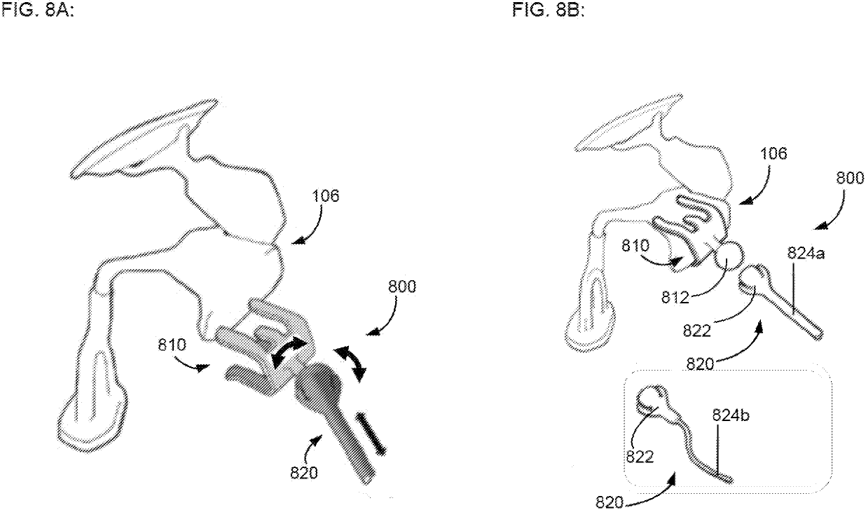

FIGS. 7A-7F schematically illustrate example second elements 430 in accordance with certain implementations described herein. FIGS. 7A and 7B schematically illustrate two example second elements 430 with the second end 434 comprising a ball having a diameter of 0.5 millimeter and 1 millimeter, respectively, the second end 434 configured to be in contact with (e.g., pressed against; affixed by adhesive to) a portion of the otic capsule, a portion of the cochlea 140 (e.g., the round window 121, the oval window 112, the promontory 123), or an ossicle 106 (e.g., the incus 109 or stapes 111) of the recipient. In certain implementations, the second end 434 of the second element 430 is configured to be osseo-integrated with a portion of the otic capsule or a portion of the cochlea 140. For example, the second end 434 of the second element 430 can be treated to facilitate osseo-integration with the portion of the cochlea 140. For another example, the second end 434 of the second element 430 can comprise a male/female portion configured to mate with a corresponding female/male fixture configured to be affixed (e.g., osseo-integrated; glued using bone cement) to a portion of the recipient’s body (e.g., cochlea 140; optic capsule; ossicle 106). In certain such implementations in which the second element 430 is configured to rotate axially, the mating of the second end 434 of the second element 430 to the corresponding female/male fixture can be performed by axially rotating the second element 430 (e.g., a rotation fit). FIG. 7C schematically illustrates an example second element 430 with the second end 434 comprising a cylinder having a diameter of 1 millimeter, the second end 434 configured to be in contact with (e.g., pressed against; affixed by adhesive to) the round window 121, oval window 112, or the footplate of the stapes 111 of the recipient. FIG. 7D schematically illustrates an example second element 430 with the second end 434 comprising a clip (e.g., a Wèngen clip) configured to grip the long process of the incus 109 or the stapedial arch or neck of the stapes 111 of the recipient. FIG. 7E schematically illustrates an example second element 430 with the second end 434 comprising a bell-shaped receptacle configured to be in contact with the crus or head of the stapes 111 of the recipient. FIG. 7F schematically illustrates an example second element 430 with the second end 434 comprising a Dresden-type clip configured to grip the capitulum of the stapes 111 of the recipient.

In certain implementations, the second end 434 of the second element 430 comprises a clip configured to be slid onto the incus 109 while the second element 430 is slidably adjusted into position within the first element 420 during implantation (e.g., for SNHL applications). Certain such implementations provide a visual indication of proper mechanical coupling of the second element 430 to the incus 109, which can be a more reliable and consistent indication of proper mechanical coupling than for systems which utilize a “rod-to-incus” pre-load approach to evaluating the mechanical coupling. In certain implementations, the clip of the second end 434 is configured to be used both for an actuator-to-incus mechanical coupling and for a microphone-to-incus mechanical coupling.

While FIGS. 4A-4B, 5A-5B, 6A-6E, and 7A-7F schematically illustrate various aspects of example medical implants (e.g., auditory prostheses) using a transducer 410 comprising an actuator and a second element 430 comprising an elongate rigid body, certain other implementations are compatible with medical implants (e.g., auditory prostheses) using a transducer 410 comprising a microphone and a second element 430 comprising an elongate flexible wire. For example, the transducer 410 can comprise a microphone assembly spaced from the ossicles 106 and the second element 430 can comprise an extension (e.g., stiff extension; flexible extension or flexible wire) affixed to a diaphragm of the microphone assembly and to an ossicle 106, the extension configured to move in response to movement of the ossicle 106. The microphone assembly can be configured to generate electrical signals in response to movement of the diaphragm caused by the movement of the extension. The electrical signals from the microphone assembly can be provided to an actuator configured to generate mechanical vibrations to another portion of the recipient’s auditory system (e.g., to bypass a malfunctioning portion of the recipient’s auditory system between the ossicle 106 and the other portion of the recipient’s auditory system).

FIGS. 8A-8B schematically illustrate an example apparatus 800 (e.g., second element 430) in accordance with certain implementations described herein. The apparatus 800 comprises a first portion 810 configured to be in contact with (e.g., attached; clipped; affixed using adhesive) to a portion of the otic capsule, a portion of the cochlea 140 (e.g., a round window, an oval window, an artificial window, a natural fenestrae, an artificial fenestrae, a promontory), a semicircular canal region, or an ossicle 106 of a recipient. The apparatus 800 further comprises a second portion 820 configured to be in mechanical communication with a transducer 410 and with the first portion 810 such that the second portion 820 is configured to be rotatably adjusted in at least one direction (e.g., in at least two directions; in two orthogonal directions) relative to the first portion 810 (the two orthogonal directions denoted in FIG. 8A by two curved double-headed arrows). In certain implementations, one of the first portion 810 and the second portion 820 comprises a male portion and the other of the first portion 810 and the second portion 820 comprises a female portion configured to engage the male portion. In certain implementations, the apparatus 800 comprises a ball joint coupler (e.g., one of the first portion 810 and the second portion 820 comprises a ball and the other of the first portion 810 and the second portion 820 comprises a socket configured to engage the ball). For example, as schematically illustrated by FIGS. 8A-8B, the first portion 810 comprises a ball 812 and the second portion 820 comprises a socket 822 engaging the ball 812. For example, an outer diameter of the ball 812 and an inner diameter of the socket 822 can be in a range of 0.2 millimeter to 2 millimeters (e.g., in a range of 0.1 millimeter to 0.3 millimeter). In certain implementations, the second portion 820 is configured to rotate about an axis extending from the first portion 810 to the second portion 820 (e.g., to rotate axially about an axis of the second element 430 that comprises the second portion 820).

As schematically illustrated by FIG. 8B, the second portion 820 of certain implementations comprises a rigid rod 824a attached to the socket 822 while the second portion 820 of certain other implementations comprises a flexible wire 824b attached to the socket 822. In certain implementations, the apparatus 800 reduces (e.g., minimizes) an amount of pre-load force applied to the ossicular chain as compared to a rigid, non-rotatably adjustable coupler. In certain implementations, the apparatus 800 reduces (e.g., minimizes) an amount of conductive losses between the apparatus 800 and the ossicular chain as compared to a rigid, non-rotatably adjustable coupler.

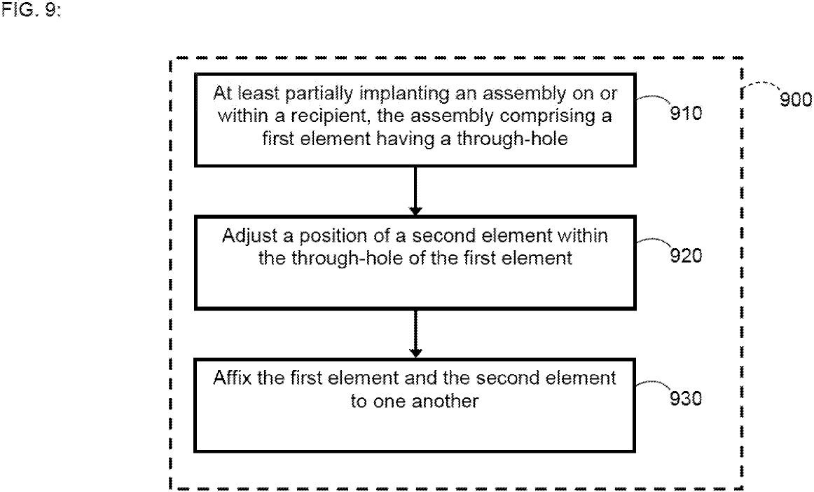

FIG. 9 is a flow diagram of an example method 900 in accordance with certain embodiments described herein. While the example method 900 is described herein by referring to the example apparatus 400, 800 of FIGS. 4A-4B, 5A-5B, 6A-6E, 7A-7F, and 8A-8B, other apparatuses are also compatible with the example method 500 in accordance with certain embodiments described herein. For example, the method 900 described herein can be applied to any of a variety of implantable medical devices.

In an operational block 910, the method 900 comprises at least partially implanting an assembly (e.g., apparatus 400) on or within a recipient such that the assembly is in mechanical communication with a first portion of a recipient’s body (e.g., skull 304). The assembly can comprise a transducer 410 and a first element 420 in mechanical communication with the transducer 410, the first element 420 comprising a through-hole (e.g., orifice 422).

In an operational block 920, the method 900 further comprises adjusting a position of a second element 430 within the through-hole such that the second element 430 extends out of both sides of the through-hole and the second element 430 is in mechanical communication with a second portion of the recipient’s body (e.g., an ossicle 106). In certain implementations, the position of the second element 430 is adjusted (e.g., slid) along an axial direction of the second element 430 (e.g., z-direction) such that the second element 430 spans a desired distance between the first element 420 and the second portion of the recipient’s body (e.g., a desired coupling distance). In certain implementations in which the through-hole comprises a slit (e.g., a slit with an integrated clip), adjusting the position of the second element 430 within the through-hole can comprise positioning the second element 430 at a selected position along the slit.

In an operational block 930, the method 900 further comprises affixing the first element 420 and the second element 430 to one another such that mechanical vibrations generated by the transducer 410 propagate through the first element 420 and the second element 430 to the second portion of the recipient’s body. In certain implementations, affixing the first element 420 and the second element 430 to one another comprises crimping a portion of the first element 420 to compress a portion of the second element 430. In certain other implementations, affixing the first element 420 and the second element 430 to one another comprises applying an adhesive to one or both of a portion of the first element 420 and a portion of the second element 430. In certain implementations, the transducer 410 is between the portion of the first element 420 that is affixed to the second element 430 and the second portion of the recipient’s body. For example, the first element 420 can be affixed to the second element 430 (e.g., by crimping and/or by applying adhesive) at a position within the fixation element 440, as schematically illustrated by FIG. 4B). In certain other implementations, the portion of the first element 420 that is affixed to the second element 430 is between the transducer 410 and the second portion of the recipient’s body. For example, the first element 420 can be affixed to the second element 430 (e.g., by crimping and/or by applying adhesive) at a position between the transducer 410 and the ossicles 106, as schematically illustrated by FIG. 5B). In certain implementations, the method 900 further comprises cutting the second element 430 after said affixing the first element 420 and the second element 430 to one another (e.g., to remove a portion of the second element 430 extending out of one side of the through-hole in a direction away from the second portion of the recipient’s body).

Certain implementations described herein provide simplified implantation procedures and/or tools, thereby reducing the probability of errors or mishaps during the transducer assembly implantation process resulting from the handling, cutting, and positioning of the extension element between the transducer and sensitive structures of the recipient’s body. For example, the manipulation of the extension element is reduced, thereby reducing the risk of losing or damaging the extension element, as well as the risk of measuring or cutting errors.

Certain implementations described herein enable various extension coupling variant options without logical complexity. For example, an extension element can be pre-slid into the transducer assembly (e.g., for SNHL), but the extension element can be easily replaced in the operating room by another extension element if desired (e.g., for MHL). Certain implementations described herein enable similar surgical procedures and configurations to be used for transducer assemblies configured to address SNHL and transducer assemblies configured to address MHL, thereby resulting in more consistent surgeries for a wider range of surgeons and more consistent outcomes.

Certain implementations described herein reduce the risk of mechanical interference (e.g., contact between the transducer assembly and surrounding bone) by having only the extension element linearly translated into position, as opposed to the transducer being moved (e.g., downward or into the middle ear cavity). In addition, transducer assemblies of certain implementations described herein do not include a linear motion mechanism (e.g., z-direction microdrive and compression unit) for moving the transducer, thereby reducing complexity and expense (e.g., only including an axial rotational degree of freedom).

Although commonly used terms are used to describe the systems and methods of certain implementations for ease of understanding, these terms are used herein to have their broadest reasonable interpretations. Although various aspects of the disclosure are described with regard to illustrative examples and implementations, the disclosed examples and implementations should not be construed as limiting. Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

It is to be appreciated that the implementations disclosed herein are not mutually exclusive and may be combined with one another in various arrangements. In addition, although the disclosed methods and apparatuses have largely been described in the context of conventional cochlear implants, various implementations described herein can be incorporated in a variety of other suitable devices, methods, and contexts. More generally, as can be appreciated, certain implementations described herein can be used in a variety of implantable medical device contexts that can benefit from having at least a portion of the received power available for use by the implanted device during time periods in which the at least one power storage device of the implanted device unable to provide electrical power for operation of the implantable medical device.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ± 10% of, within ± 5% of, within ± 2% of, within ± 1% of, or within ± 0.1% of the stated amount. As another example, the terms “generally parallel” and “substantially parallel” refer to a value, amount, or characteristic that departs from exactly parallel by ± 10 degrees, by ± 5 degrees, by ± 2 degrees, by ± 1 degree, or by ± 0.1 degree, and the terms “generally perpendicular” and “substantially perpendicular” refer to a value, amount, or characteristic that departs from exactly perpendicular by ± 10 degrees, by ± 5 degrees, by ± 2 degrees, by ± 1 degree, or by ± 0.1 degree. The ranges disclosed herein also encompass any and all overlap, sub-ranges, and combinations thereof. Language such as “up to,” “at least,” “greater than,” less than,” “between,” and the like includes the number recited. As used herein, the meaning of “a,” “an,” and “said” includes plural reference unless the context clearly dictates otherwise. Also, as used in the description herein, the meaning of “in” includes “into” and “on,” unless the context clearly dictates otherwise.

While the methods and systems are discussed herein in terms of elements labeled by ordinal adjectives (e.g., first, second, etc.), the ordinal adjective are used merely as labels to distinguish one element from another (e.g., one signal from another or one circuit from one another), and the ordinal adjective is not used to denote an order of these elements or of their use.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein, but should be defined only in accordance with the claims and their equivalents.

What is claimed is:

1. An apparatus comprising:
a transducer configured to be at least partially implanted on or within a recipient and in mechanical communication with a first portion of the recipient’s body;
a first element configured to be at least partially implanted on or within the recipient, the first element configured to be in mechanical communication with the transducer, the first element comprising an orifice having a first length and extending from a first surface of the first element to a second surface of the first element; and
a second element configured to be at least partially implanted on or within the recipient, the second element comprising a first end and a second end with a second length therebetween, the second length longer than the first length, the second element configured to extend through the orifice and, during implantation, to be slidably adjusted relative to the orifice and affixed to the first element such that the second end is in mechanical communication with a second portion of the recipient’s body.

2. The apparatus of claim 1, wherein the first element comprises a tube extending through the transducer, the orifice comprises a hollow region within the tube.

3. The apparatus of claim 1, wherein the orifice comprises a through hole extending from the first surface to the second surface.

4. The apparatus of claim 2, wherein the second element comprises a rod.

5. The apparatus of claim 2, wherein the second element comprises a wire.

6. The apparatus of claim 1, wherein the second end comprises a clip configured to be attached to the second portion of the recipient’s body.

7. The apparatus of claim 1, wherein the transducer comprises an actuator configured to generate mechanical vibrations that, upon the second element being affixed to the first element, propagate through the first element and the second element to the second portion of the recipient’s body.

8. The apparatus of claim 1, wherein the transducer comprises a microphone configured to receive mechanical vibrations that, upon the second element being affixed to the first element, propagate from the second portion of the recipient’s body, through the first element and the second element, to the microphone.

9. The apparatus of claim 1, further comprising a fixation element configured to be affixed to the first portion of the recipient’s body and in mechanical communication with the transducer such that an angular orientation of the transducer relative to the fixation element is configured to be controllably adjusted.

10. The apparatus of claim 9, wherein the fixation element comprises an adjustably rotatable coupler.

11. The apparatus of claim 9, wherein the fixation element comprises an adjustably bendable coupler.

12. The apparatus of claim 1, wherein the first element is configured to be affixed to the second element at one or more positions along the first element.

13. The apparatus of claim 12, wherein the one or more positions comprises a position between the first end of the second element and the transducer.

14. The apparatus of claim 12, wherein the one or more positions comprises a position between the second end of the second element and the transducer.

15. The apparatus of claim 1, wherein the first portion of the recipient’s body comprises a portion of the recipient’s skull and the second portion of the recipient’s body comprises an ossicle of the recipient’s body.

16. The apparatus of claim 1, wherein at least a portion of the first element and at least a portion of the second element are configured to be implanted within a middle ear region of the recipient’s body, and the second end of the second element is configured to be in mechanical communication with an ossicle, a portion of the cochlea, or a semicircular canal of the recipient’s body.

17. An apparatus comprising:
a first portion configured to be in contact with a portion of a recipient’s auditory system; and
a second portion configured to be in mechanical communication with a transducer and with the first portion such that the second portion is configured to be rotatably adjusted in at least one direction relative to the first portion.

18. The apparatus of claim 17, wherein the second portion is configured to be rotatably adjusted in at least two directions relative to the first portion.

19. The apparatus of claim 17, wherein one of the first portion and the second portion comprises a male portion and the other of the first portion and the second portion comprises a female portion configured to engage the male portion.

20. The apparatus of claim 17, wherein one of the first portion and the second portion comprises a ball and the other of the first portion and the second portion comprises a socket configured to engage the ball.

21. The apparatus of claim 17, wherein the first portion is configured to be in contact with a portion of the otic capsule, a portion of the cochlea, a semicircular canal region, or an ossicle of the recipient.

22. The apparatus of claim 21, wherein the portion of the cochlea comprises a round window, an oval window, an artificial window, or a promontory.

23. The apparatus of claim 21, wherein the ossicle comprises an incus or a stapes of the recipient.

24. The apparatus of claim 17, wherein the second portion is configured to rotate about an axis extending from the first portion to the second portion.

25. A method comprising:
at least partially implanting an assembly on or within a recipient such that the assembly is in mechanical communication with a first portion of a recipient’s body, the assembly comprising a transducer and a first element in mechanical communication with the transducer, the first element comprising a through-hole;
adjusting a position of a second element within the through-hole such that second element extends out of both sides of the through-hole and the second element is in mechanical communication with a second portion of the recipient’s body; and
affixing the first element and the second element to one another such that mechanical vibrations generated by the transducer propagate through the first element and the second element to the second portion of the recipient’s body.

26. The method of claim 25, wherein affixing the first element and the second element to one another comprises crimping a portion of the first element to compress a portion of the second element.

27. The method of claim 25, wherein affixing the first element and the second element to one another comprises applying an adhesive to one or both of a portion of the first element and a portion of the second element or clipping the first element and the second element to one another.

28. The method of claim 25, further comprises axially rotating the second element to mechanically couple the second element to a fixture affixed to the second portion of the recipient’s body.

29. The method of claim 25, wherein the transducer is between a position at which the first element is affixed to the second element and the second portion of the recipient’s body.

30. The method of claim 25, wherein a position at which the first element is affixed to the second element is between the transducer and the second portion of the recipient’s body.

31. The method of claim 25, wherein the first element extends through the transducer.
